(19) United States
(12) Patent Application Publication
(10) Pub. No.: US 2003/0059858 A1
(43) Pub. Date: Mar. 27, 2003

(54) Functional screening for transcription factors

(75) Inventors: Christoph Wiesner, Wien (AT); Martina Hoeth, Wien (AT); Rainer De Martin, Wien (AT)

Correspondence Address: THE FIRM OF KARL F ROSS, 5676 RIVERDALE AVENUE, PO BOX 900, RIVERDALE (BRONX), NY, 10471-0900, US

(73) Assignee: BMT Medizinische Forschung und Entwicklung GmbH

(21) Appl. No.: 10/254,040

(22) Filed: Sep. 24, 2002

(30) Foreign Application Priority Data
Sep. 27, 2001 (AT) A1538/2001

(51) Int. Cl. G01N 33/567; G01N 33/53; G01N 33/569; G06F 19/00; G01N 33/48; G01N 33/50

(52) U.S. Cl. 435/7.21; 435/7.31; 702/19

(57) ABSTRACT

A method to specifically identify transcription factors and transcriptional coactivators from a given cell type and the use of individual clonal cell lines stable expressing gal4-TA domain hybrid proteins for screening or characterization of novel drugs as well as of mixed cell populations stably expressing different gal4-TA domain hybrid proteins for high-throughput screening of novel drugs.

### INTRODUCTION

Transcription factors (TF) control the expression of genes and thus are key regulatory molecules for a wide variety of cellular functions, representing valuable targets for therapeutic interference. They consist usually of a DNA binding (DB), a transactivation (TA) and sometimes additional, such as dimerization, ligand binding, domains. Gene expression is achieved by binding to sequence-specific sites in the respective promoter regions and interaction with the basal transcription machinery.

Although functional annotation of sequences from the human and other sequencing projects has been successful through identifying a wide variety of protein domains, this has been difficult to achieve for TFs. Concerning DNA binding domains (BDs), although some of them such as the homeodomain or fork-head (Weigel et al., 1990; Miller et al., 1994) domains are indicative for TFs, others like helix-turn-helix or leucine zipper may also mediate protein-protein interactions. On the other hand, TA domains are even more poorly defined. Therefore a genetic screen has been developed to identify TFs by virtue of their TA properties, followed by their analysis in regard to the presence of a potential DNA binding domain (BD).

### IDENTIFICATION OF TFs

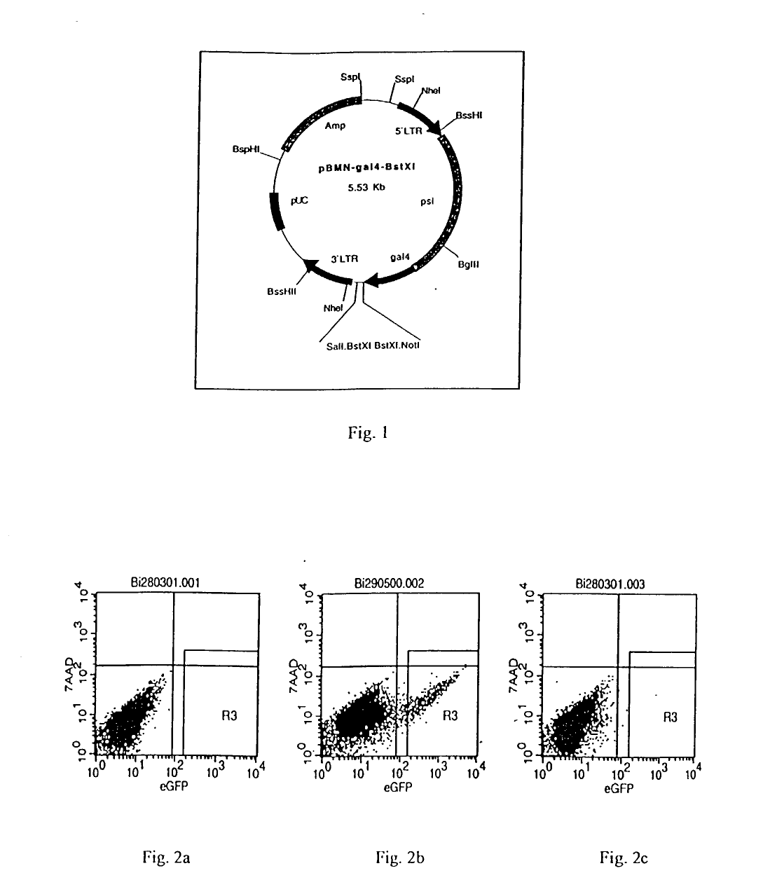

The mammalian reverse one-hybrid system. The classical yeast one hybrid system is a method to identify TFs that bind to a cognate (multimerized) DNA binding site (Wei et al., 1999). It uses a cDNA library that expresses inserts as fusion genes with the gal4 TA domain, leading to yeast growth on selective media in the case that the cDNA insert contains the DNA binding domain recognizing the respective DNA sequence. Vice versa, a cDNA library in a vector has been generated that expresses inserts as fusion genes with the yeast Gal4 DNA binding domain (FIG. 1) and adopted to mammalian cells. The method is thus characterized by a plasmid vector which expresses cDNA inserts as fusion proteins with the yeast Gal4 DNA binding domain; it is further characterized by an appropriate reporter cell line which contains a Gal4 dependent promoter reporter gene so that cDNA inserts containing a transactivation domain form together with the Gal4 DNA binding domain from the vector a functional transcription factor which effects the expression of the reporter gene.

Thus it is a general method to identify transcription factors independent of their DNA binding sequence as well as co-activators. It is therefore referred to as “mammalian reverse one-hybrid system”. The method thus differs from the yeast one-hybrid system among others in that in the latter specific transcription factors which bind exclusively to that given sequence are identified by a cognate DNA sequence.

FIG. 1 is a map of the retroviral vector pBMN-gal4-BstXI. A retroviral vector termed pBG4B was constructed that is based on pBABE (Morgenstern and Land, 1990) and contains the yeast gal4 DNA BD upstream of a polylinker containing two BstXI sites and cDNA from cytokine-stimulated EC inserted. After packaging into retroviral particles, the library was used to transduce a murine NIH3T3-based reporter cell line that has been stable transfected with a gal4-dependent EGFP (enhanced green fluorescent protein; Tsien 1998) reporter gene. cDNAs that encode a functional TA domain activate the reporter gene, and EGFP-positive cells are isolated by FACS and expanded, allowing the isolation and analysis of the integrated inserts.

The screening method is characterized by the following steps:

1. Generation of a reporter cell line by stable transfection of a gal4 dependent promoter-EGFP reporter construct into NIH3T3 cells.

2. Isolation of mRNA from cytokine-simulated EC

3. Synthesis of cDNA

4. Ligation into pBMN-gal4-BstXI using BstXI adapters

5. Transformation into electrocompetent E.coli

6. Preparation of plasmid DNA

7. Transfection of plasmid DNA into Phoenix eco retroviral packaging cells

8. Collection of retrovirus supernatant and infection of NIH3T3-gal4 reporter cells

9. Sorting of GFP-positive cells by FACS

10. Expansion of sorted cells, isolation of mRNA, and isolation of cDNA inserts by RT-PCR, sequencing

This concept was tested first by using the NF-kB p65 TA domain (Schmitz et al., 1994) inserted into the retroviral vector as positive control. After retroviral transduction, EGFP positive cells could readily be distinguished from control cells by FACS analysis, see FIGS. 2a, 2b, 2c referring to FACS sorting of GFP positive cells where FIG. 2a relates to negative control (non-infected reporter cells); FIG. 2b relates to positive control (reporter cells transduced with NF-kB p65 TA in pBMN-gal4-BstXI; FIG. 2c relates to reporter cells transduced with the retroviral library. A score of 1 positive per 20.000 cells in the FACS analysis was found to correspond to a single integration in 90% of the positive clones (FIGS. 2a, 2b, 2c). Sorted cells were expanded for further analysis. We found that RT-PCR was superior to PCR from genomic DNA to re-isolate integrated cDNAs. Subsequent sequence analysis of inserts from positive clones revealed the identity of the isolated clones, which is given in Tab. 1.

TABLE 1
Examples for TFs isolated by our screening system:

| NAME | GenBank Accession Nr. |
|---|---|
| MAML-1 | AF221759 |
| Notch-1 | AF308602 |
| HSF-1 | HUMHSF1 |
| Sox-7 | BC004299 |
| Eric-1 | HSA243997 |
| MondoA | AF312918 |
| E7 | XM_018098 |
| STAT6 | XM_043112 |
| Fus | HSFUSA |

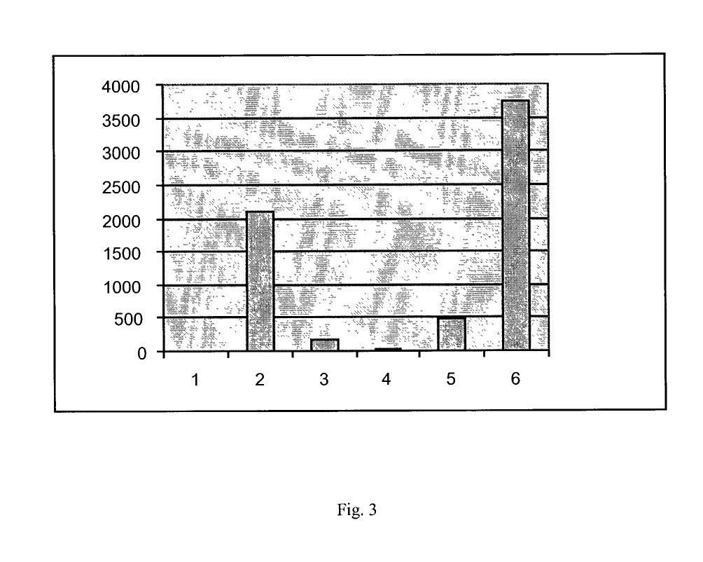

Analysis of TA domains and confirmation of the transactivating properties. Database searches revealed the identity of the isolated clones. A number of cognate TFs was identified (see Tab. 1). The inserts from selected clones were cloned into the pBMN-Gal4-BstXI vector, and transfected into cells together with pFR-Luc (a gal4 dependent promoter-luciferase reporter construct; Stratagene) to re-evaluate their TA potential. The results are shown in FIG. 3.

FIG. 3 relates to Luciferase assay. cDNA inserts recovered by RT-PCR were subcloned back into the pBMN-Gal4-BstXI vector and transiently transfected into NIH3T3 cells together with the Gal4-Luc reporter as well as RSV-βgal as internal control. Luciferase values were normalized for β-galactosidase expression. 1 (negative control): empty pBMN-Gal4-BstXI vector; 2 (positive control): NF-kB p65 TA domain in pBMN-Gal4-BstXI; 3: MAML-1; 4: STAT6; 5: E7; 6: Sry7.

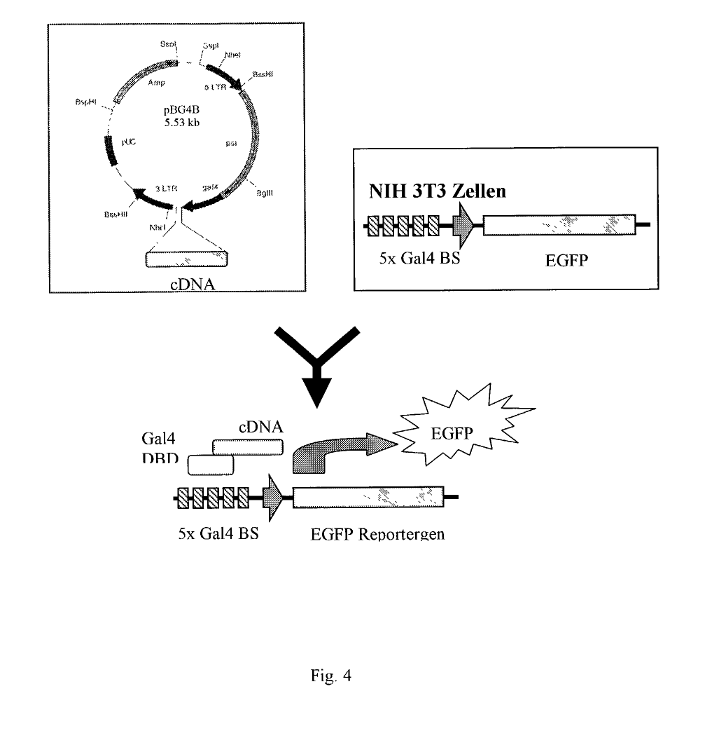

FIG. 4 is a diagram showing the steps of the screening method.

The following examples substantially illustrate the subject of the invention:

### EXAMPLE 1

A cDNA bank is generated from mRNA from cytokine-stimulated (a mix of TNFα, IL-1 and LPS, applied for a period of 0, 2 and 6 hours) endothelial cells in the pBG4G plasmid vector. That vector is a derivative of the retroviral expression vector pBABE and additionally contains the yeast GAl4 DNA binding domain such that cDNA inserts are expressed as fusion proteins with the gal4 DNA binding domain. After packaging into retroviral particles the bank is transduced into a reporter cell line. That reporter cell line consists of NIH3T3 cells into which a reporter gene has been stably integrated. The reporter gene comprises a Gal4 dependent promoter controlling the expression of GFP. cDNA inserts containing a DNA binding domain form together with the Gal4 DNA binding domain from the vector a functional transcription factor which effects the expression of the reporter gene which can be detected by fluorescence microscopy or by FACS. Thus cells which are detected by their fluorescence are isolated by FACS, expanded, the cDNA insert isolated by RT-PCR and sequenced.

### EXAMPLE 2

Individual clonal cell lines containing a particular fusion protein consisting of the yeast Gal4 DNA binding domain as well as of the transactivation domain of a particular transcription factor may be used to identify low-molecular substances which affect the transactivating activity of that transcription factor. For this purpose chemical substances of different concentrations, e.g. from a combinatorial bank, are added to the respective cell line and after an appropriate period of time the expression of the reporter gene is measured and compared with the expression before addition of the substance.

### EXAMPLE 3

Different cell lines each containing a particular fusion protein consisting of the yeast Gal4 DNA binding domain as well as of the transactivation domain of a particular transcription factor may be used to explain the mechanism of the effect of a low-molecular substance or of a drug. For this purpose the substance to be tested is added at different concentrations to the different cell lines and the expression of the reporter genes is measured and compared with the expression before addition of the substance.

### EXAMPLE 4

The method may be employed to identify transactivation domains of different strengths. Transactivation domains of different strengths are needed to generate engineered transcription factors which are employed in gene therapeutic methods for modulating the expression of specific genes. With the method described e.g. isolated transactivation domains may be combined as fusion proteins with engineered zinc finger DNA binding domains which bind to specific DNA binding sites in the promoter region of the genes to be influenced.

### MATERIALS AND METHODS

Construction of retroviral vector and cDNA library. A cDNA library was generated from poly(A⁺) RNA isolated from HUVEC that had been stimulated with a mixture of TNFα, IL-1α and LPS for 0, 3 and 9 hours, using an oligo random priming kit (Stratagene) followed by ligation of BstXI adapters (Seed and Aruffo, 1987). The size selected cDNA (>1.5 kb) was ligated into the retroviral vector pBABE (Morgenstern and Land, 1990) that had been modified by insertion of the yeast gal DNA BD followed by a polylinker containing two non-self complementary BstXI sites (pBMN-Gal4-BstXI). The ligation was transformed into XL-10 electrocompetent cells (Stratagene) yielding a library of 2.2×10₇ independent clones, 90% of which had inserts of an average size of 1.3 kb. The library was packaged into retroviral particles using the Phoenix eco packaging cell line (Hitoshi et al., 1998). As positive control, the NF-kB p65 TA domain (Schmitz et al., 1994) was inserted into the same vector.

Construction of a gal4 promoter dependent GFP reporter cell line. Murine NIH3T3 cells were transfected with a vector (pFR-EGFP) containing the gal4-dependent minimal promoter from pFR-Luc (Stratagene) driving EGFP. Cells were selected with puromycin and several clones tested in transient transfections for EGFP expression using the gal4-NF-kB TA construct as positive control.

Retroviral transduction, FACS sorting and analysis. Retroviral SN was applied to the reporter cell line as described and EGFP positive clones sorted by FACS directly into 96 well plates using a FACS Vantage (Becton Dickinson Immunocytometry Systems, San Jose, Calif., USA) FACS sorter. RNA was isolated from individual clones 14 days later and inserts recovered by RT-PCR and sequenced.

Luciferase assay. PCR fragments from positive clones were digested with BstXI and inserted into the pBMN-Gal4-BstXI, and transfected into cells together with pFR-Luc as well as RSV-βgal as internal control. Luciferase and 1-gal assays were performed as described (de Wet et al., 1987).

The disclosure and content of the following references is included in this application by reference.

### REFERENCES

Hitoshi, Y., Lorens, J., Kitada, S. I., Fisher, J., LaBarge, M., Ring, H. Z., Francke, U., Reed, J. C., Kinoshita, S., and Nolan, G. P. Toso, a cell surface, specific regulator of Fas-induced apoptosis in T cells. Immunity. 1998;8:461-71.

Miller, C. P., McGehee, R. E., Jr., and Habener, J. F. IDX-1: a new homeodomain transcription factor expressed in rat pancreatic islets and duodenum that transactivates the somatostatin gene. Embo J. 1994;13:1145-56.

Morgenstern, J. P., and Land, H. Advanced mammalian gene transfer: high titre retroviral vectors with multiple drug selection markers and a complementary helper-free packaging cell line. Nucleic Acids Res. 1990;18:3587-96.

Schmitz, M. L., dos Santos Silva, M. A., Altmann, H., Czisch, M., Holak, T. A., and Baeuerle, P. A. Structural and functional analysis of the NF-kappa B p65 C terminus. An acidic and modular transactivation domain with the potential to adopt an alpha-helical conformation. J Biol Chem. 1994;269:25613-20.

Seed, B., and Aruffo, A. Molecular cloning of the CD2 antigen, the T-cell erythrocyte receptor, by a rapid immunoselection procedure. Proc Natl Acad Sci USA. 1987;84:3365-9.

Tsien, R. Y. The green fluorescent protein. Annu Rev Biochem. 1998;67:509-44.

Wei, Z., Angerer, R. C., and Angerer, L. M. Identification of a new sea urchin ets protein, SpEts4, by yeast one-hybrid screening with the hatching enzyme promoter. Mol Cell Biol. 1999;19:1271-8.

Weigel, D., Seifert, E., Reuter, D., and Jackle, H. Regulatory elements controlling expression of the Drosophila homeotic gene fork head. Embo J. 1990;9:1199-207.

de Wet, J. R., Wood, K. V., DeLuca, M., Helinski, D. R., and Subramani, S. Firefly luciferase gene: structure and expression in mammalian cells. Mol Cell Biol. 1987;7:725-37.

1. A method to specifically identify transcription factors and transcriptional coactivators from a given cell type; the method may used in mammalian cells, but also in other cell types, such as yeast.

2. The use of individual clonal cell lines stable expressing gal4-TA domain hybrid proteins for screening or characterization of novel drugs.

3. The use of mixed cell populations stably expressing different gal4-TA domain hybrid proteins for high-throughput screening of novel drugs.

4. The use of this system for the identification of strong TA domains as components of in engineered TFs.
